(19) United States
(12) Patent Application Publication
(10) Pub. No.: US 2003/0220613 A1
(43) Pub. Date: Nov. 27, 2003

(54) Syringe, especially a pre-filled one-time-use syringe, and method of manufacturing same

(76) Inventors: Arthur Fabian, Schaffhausen (CH); Horst Koller, Engelburg (CH); Ralf Holschumacher, Engelburg (CH); Bertram Eichhorn, Engelburg (CH); Thomas Kadur, Engelburg (CH)

Correspondence Address: STRIKER, STRIKER & STENBY, 103 East Neck Road, Huntington, NY, 11743, US

(21) Appl. No.: 10/443,706

(22) Filed: May 22, 2003

(30) Foreign Application Priority Data
May 25, 2002 (DE) 102 23 421.3

(51) Int. Cl. A61M 5/00

(52) U.S. Cl. 604/187

(57) ABSTRACT

The syringe, especially a pre-filled one-time-use syringe, has a syringe body, which includes a cylindrical supply chamber for a liquid to be injected and a piston with a piston rod axially connected to it. A finger support is provided at one end of the syringe body and an end portion for holding a hollow needle is provided at the other end of the syringe body. The hollow needle is clamped in a needle channel provided in the end portion. A method of making a syringe body is described, in which the cylindrical storage chamber is made in one piece with a threaded connector by an injection molding process, the end portion provided with the hollow needle clamped in it is made in another injection molding process and then after making the end portion and the cylindrical storage chamber the cylindrical storage chamber is connected with the end portion.

### BACKGROUND OF THE INVENTION

1. Field of the Invention

The present invention relates to a syringe, especially a pre-filled one-time-use syringe made of glass or plastic, with a syringe body, which comprises a cylindrical storage chamber for a liquid to be injected, and with a piston and piston rod axially connected to it, so that the piston is axially slidable in the storage chamber, wherein the syringe body is associated with a finger support at one end and an end portion for holding a hollow needle at another end opposite the one end.

2. Description of the Related Art

Syringes, especially one-time-use syringes, are known in many forms and are used also as pre-filled one-time-use syringes, which can have a glass syringe body or especially a plastic syringe body. These known one-time-use syringes include a syringe body, which has a cylindrical supply chamber for a liquid to be injected, a piston and a piston rod connected with it so that the piston is axially slidable. The syringe body is associated with a finger support provided at one end and an end portion opposite to the finger support for holding a hollow needle, which is also sometimes called a cannula. In the known syringe the hollow needle is arranged in a needle channel provided in the end portion. The hollow needle is fixed in the needle channel by gluing it in the needle channel in the end portion of the syringe.

The known syringe however has the disadvantage that the adhesive used can come into contact with the liquid to be injected because of the gluing of the hollow needle with the end portion, which has the consequence that the adhesive used must be permissible and must fulfill the legal requirements, which limit, on the one hand, the choices for selection from possible adhesives and, on the other hand, cause considerable effort before and during manufacture of the syringe.

DE 29 39 180 A1 discloses an ampoule one-time-use injector syringe, in which the supply chamber contains a medicine and in which the nosepiece receiving the hollow needle is made separately from the supply chamber and subsequently assembly with it. The hollow needle is glued with the nosepiece.

DE 44 38 360 A1 discloses a pre-filled one-time-use syringe with a filling volume less than 5 ml. The syringe body with syringe cylinder, gripping member and syringe head is formed from glassy transparent plastic with very small gas permeability and resistance to gamma radiation and/or ethylene oxide gas. The syringe needle is integrated directly in the plastic syringe head. This integration can occur so that the region of the syringe head, in which the syringe needle is held, is embodied as an extrusion coating of the needle.

### SUMMARY OF THE INVENTION

It is an object of the present invention to provide a syringe or an injector of the above-described type, which has an especially simple structure.

It is an object of the present invention to provide a syringe or an injector of the above-described type, especially a pre-filled one-time-usage syringe, which has an especially simple structure and which does not have the above-described disadvantages.

These objects and others, which will be made more apparent hereinafter, are attained in a syringe, especially a pre-filled one-time-use syringe made of glass or plastic, comprising a syringe body which includes a cylindrical storage chamber for a liquid to be injected and a piston with a piston rod axially connected to it, so that the piston is axially slidable in the storage chamber, wherein the syringe body is associated with a finger support at one end and an end portion for holding a hollow needle in a needle channel at another end opposite from the one end.

According to the invention the hollow needle is held clamped or held fixed in the needle channel provided in the end portion.

The syringe according to the invention, especially the one-time-use syringe, has a hollow needle clamped in the needle channel in the end portion instead of being glued in the end section, as in the prior art. An adhesive is not used to fix the hollow needle in the end portion in the syringe according to the invention. In other words the syringe according to the invention, which is embodied preferably in a one-time-usage syringe, avoids the use of an adhesive, which is required to attach the hollow needle or cannula in the case of the conventional syringe. The hollow needle according to the invention is held clamped in the end section between the appropriate part of the needle channel and the outer hollow needle wall.

The syringe according to the invention has a series of advantages. First because of the clamping of the hollow needle in the end portion without the use of adhesive the manufacture of the syringe according to the invention is considerably simplified. The additional process steps in which the region receiving the adhesive is constructed in the end portion and the adhesive introduced into it are eliminated. Also no adhesive hardening time is required during the making of the syringe according to the invention, which speeds up the making of the syringe. Moreover the syringe according to the invention completely avoids adhesive that is required with the conventional syringe so that accordingly no legal requirements regarding the use of adhesive need to be considered. In that regard the syringe according to the invention is universally usable.

In order to provide a syringe that can be used over and over again, but also a one-time-use syringe, a first preferred embodiment of the syringe according to the invention provides that the hollow needle is clamped in the needle channel so that it can be removed only with a force greater than 30 N and preferably only with forces between 60 N and 120 N. This preferred embodiment guarantees that the hollow needle is not loosened in an undesirable manner from the syringe body and also so that it cannot remain in a patient when the syringe is used to inject the liquid into the patient.

So that the size of the clamping engagement of the hollow needle in the needle channel can be varied in another preferred embodiment of the syringe according to the invention an end section of the hollow needle is clamped with only a part or section of the needle channel near the storage chamber. In other words the hollow needle is only clamped with a limited part of its entire axial length in the needle channel so that accordingly in this embodiment of the syringe the needle channel of the syringe body extends cylindrically in order to guarantee the required clamping engagement with the hollow needle. A preferably conically widened region is connected with this cylindrical region of the needle channel, which makes the guiding of the hollow needle into the needle channel easier and especially into the cylindrical region.

In another preferred embodiment of the syringe according to the invention the needle channel clamps the hollow needle over its entire axial extent so that the hollow needle is reliably held securely over the entire axial length of its end section.

In order to bring about the above-described clamping engagement between the hollow needle and the needle channel in the syringe according to the invention, there are two fundamentally different possibilities.

The first possibility provides that the interior diameter of the needle channel is adjusted to fit the outer diameter of the hollow needle so that the above-described clamping engagement of the hollow needle in the needle channel is brought about. To accomplish this especially the needle channel inner diameter is about 2% to 15%, preferably from 4% to 8%, less than the outer diameter of the hollow needle, depending on the elasticity and deformability of the end portion.

The second possibility provides that the needle channel region, in which the hollow needle is clamped, has an inner diameter, which corresponds at least to the outer diameter of the hollow needle, so that the hollow needle is insertable without a great effort in this needle channel end section. Preferably however the needle channel diameter is about 2% to 10% larger than the outer diameter of the hollow needle, whereby the insertion of the hollow needle in the needle channel is speeded. Subsequently in this embodiment the end portion comprising a thermoplastic material is heated to such an extent that this end portion is deformable, so that the desired clamping engagement between the needle channel wall and the hollow needle arranged in it is brought about by deforming the end portion at the same time.

An additional fixing of the hollow needle in the end portion can still be achieved with the above-described possibilities so that the hollow needle is inserted in the needle channel so that it extends axially into the cylindrical syringe body for a comparatively short distance. By a connected preferably radially widened part of this end of the needle extending axially into the syringe body, only destruction of the end portion can free the needle from the needle channel. Thus in this embodiment the hollow needle is fixed, not only in the needle channel, but also by the above-described widened part.

In an especially suitable embodiment of the syringe according to the invention the end portion provided with the needle channel, in which the hollow needle is clamped in the needle channel, is not formed in one piece with the syringe body or supply chamber, but is connected with the cylindrical supply chamber by a connector, so that the end portion provided with the hollow needle is made separately from the cylindrical supply chamber for the liquid to be injected. The locking of the end portion provided with the hollow needle on the cylindrical supply chamber occurs then by the connector provided on the end portion and/or on the cylindrical supply chamber. This embodiment of the syringe according to the invention has the additional advantage that the manufacture of it is further simplified, as is explained still in the following in the process according to the invention.

Basically in the above-described embodiment of the syringe according to the invention the connector can be formed arbitrarily, in so far as it guarantees that the end portion provided with the hollow needle is securely connected with the cylindrical supply chamber. It is especially advantageous, when the connecting element or connector is formed as a threaded connecting element of a screw connection, such that especially the end portion is provided with a suitable exterior thread so that it can be screwed into a suitable connecting region equipped with an interior threaded region provided in the supply chamber. Understandably it is also possible that a connection region provided on the supply chamber at one end is provided with an exterior thread and the end portion is provided with an interior thread so that it can be screwed on the exterior thread provided on the supply chamber.

In order to cover the hollow needle from the exterior exposed in the above-described embodiments of the syringe according to the invention, other embodiments of the syringe according to the invention provide the end portion with a retaining or holding region for releasably securing a needle-protecting member on its outer periphery. This retaining or holding region is preferably provided with an exterior thread so that the needle-protecting member, also called a needle shield, can be screwed onto it.

The present invention also includes a method for making a syringe body for the above-described embodiments of the syringe according to the invention. Accordingly the method of making the syringe body of the syringe according to the invention comprises making the cylindrical storage chamber in a first process step, making the end portion provided with the hollow needle clamped therein in an additional process step and then, after making the end portion and the cylindrical storage chamber, connecting the cylindrical storage chamber with the end portion to form the syringe body in a final process step. In other words the method according to the invention includes two partial process steps separate from each other, in which the first partial process step comprises making the cylindrical storage chamber and the additional different partial process steps comprise making the end portion in which the hollow needle is clamped in its needle channel. Then in the following final process step the cylindrical supply chamber is connected with the end portion having the hollow needle, so that a syringe body results, which then only needs to be provided with the piston and the piston rod connected with it and, if necessary, with a finger support or finger gripping member on one end. The method of making the syringe body can also be performed however so that the cylindrical supply chamber can be made already with the finger support on its one end.

If the method according to the invention is used to make a one-time-use syringe, especially from plastic, the above-described process steps can be performed by injection molding, so that a threaded section formed in one piece with the supply chamber and the supply chamber can be made in the first working step by this injection molding process, while in the other working steps following the first wording step the end portion is made with the hollow needle clamped in the needle channel similarly by injection molding.

The essential advantage of the methods according to the invention is especially that it is simpler to mount the hollow needle in the injection-molding tool for making the end portion because of the mold geometry. The making of the cylindrical supply chamber itself then is performed quickly and simply in the separation process step using the relatively simple injection molding tools. In case it is desired the suitable injection molding tool for making the supply chamber can be formed so that a finger support or gripping member is formed at the same time as the supply chamber at the one end.

The syringe body made by the method according to the invention may then be processed further by inserting a piston into the cylindrical supply chamber and a piston rod connected with it to form a one-time-use syringe, in which the hollow needle may then be additionally protected with a releasable or detectable needle cover (needle shield).

Advantageous further features of the syringe according to the invention and of the above-described method of making it are given in the dependent claims appended hereinbelow.

### BRIEF DESCRIPTION OF THE SEVERAL VIEWS OF THE DRAWING

The objects, features and advantages of the invention will now be described in more detail with the aid of the following description of the preferred embodiments, with reference to the accompanying figures in which:

FIGS. 1a, 1b and 1c are respective schematic cutaway cross-sectional views through a syringe according to a first embodiment of the invention during corresponding different stages in its manufacture; and

FIGS. 2a and 2b are respective schematic side and cross-sectional views through unconnected parts of a syringe according to a second embodiment of the invention prior to assembly.

### DESCRIPTION OF THE PREFERRED EMBODIMENTS

The same parts in the embodiments of the syringe according to the invention are labeled with the same reference numbers.

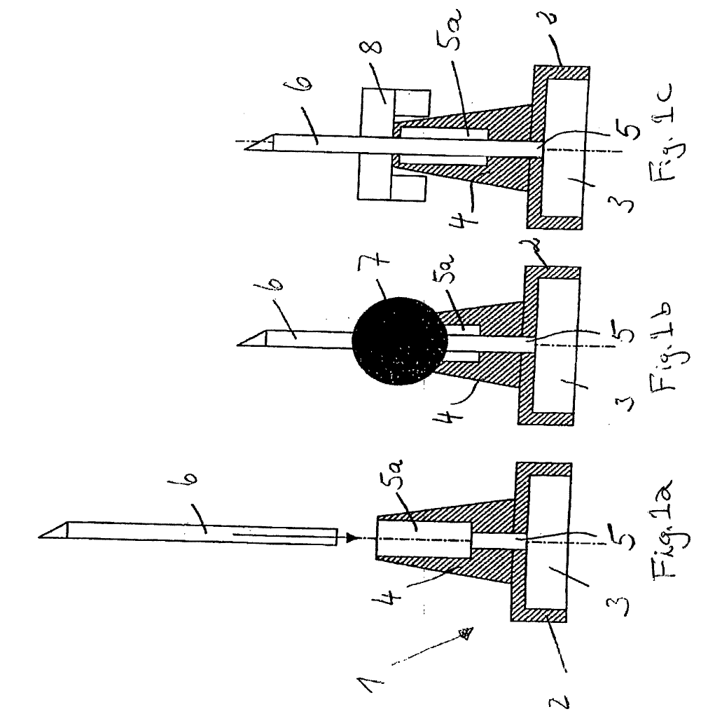

In FIG. 1a the first embodiment of the syringe is designated with the drawing reference number 1. The respective different stages in the manufacture of the syringe are again reflected in the different views of FIGS. 1a, 1b and 1c.

The syringe 1 shown in FIG. 1a has a syringe body 2, which includes a cylindrical storage chamber 3 for receiving a piston that is not shown in the drawing and a piston rod connected with the piston so that the piston is axially slidable (also not shown). A finger support or finger-gripping member, which is not shown in FIGS. 1a to 1c, is provided on one end of the syringe body 2. The syringe 1 can be securely held during its use by means of the finger support or finger-gripping element. The syringe has an end portion 4 on the end opposite from or remote from the finger support, in which a needle channel 5 is provided, in which a hollow needle 6 is inserted, as shown in FIG. 1a. For this purpose the needle channel 5 has a cylindrically widened section 5a at its outside end. This cylindrically widened section 5a receives an adhesive only in the known conventional syringes, in order to hold the hollow needle 6 in the end portion 4.

The cylindrically widened section 5a of the needle channel 5 is shown proportionately larger than it actually is in the illustrated embodiment in FIGS. 1a to 1c, in order to better illustrate the differences in diameters of the needle channel 5 and its widened section 5a.

After insertion of the hollow needle 6 in the needle channel 5, as shown in FIG. 1b, the end portion 4 of the syringe or syringe body is heated in the vicinity of the widened section 5a, as illustrated symbolically by the dark circle 7 in FIG. 1b.

Since the end portion 4 is made from thermoplastic material, the heating of the end section makes it deformable by a suitable tool 8, as shown in FIG. 1c. The cylindrically widened section 5a can be eliminated in this way. The plastic material then contacts and surrounds the hollow needle 6 inserted in the needle channel 5 on all sides. Thus the hollow needle 6 is firmly clamped in the needle channel 5 and especially in the widened section 5a.

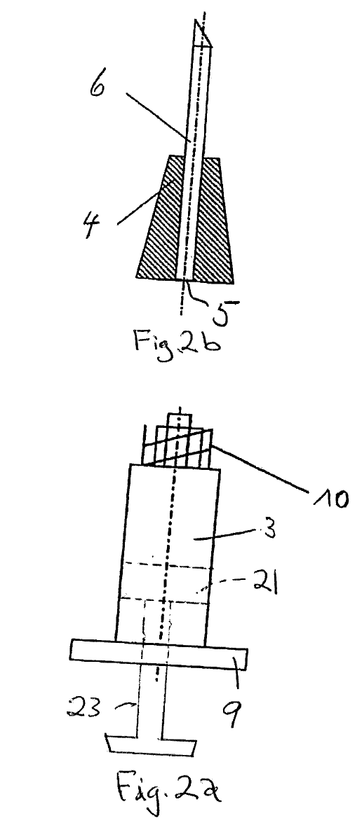

The embodiment shown in FIGS. 1a to 1c thus is a syringe, in which the end portion 4 is formed in one piece with the cylindrical supply chamber 3. In the additional embodiment of the syringe 1 shown in FIGS. 2a and 2b, the cylindrical supply chamber 3 is connected with the end portion 4 by means of a connector 10 formed in one piece with the cylindrical supply chamber 3. The connector 10 is threaded to form a screw connection between the end portion 4 and the supply chamber 3. For this purpose prior to its assembly the syringe shown unassembled in FIGS. 2a and 2b has an end section 4, which is provided with a needle channel 5, in which a hollow needle 6 is clamped.

For manufacture of this end portion it is preferred that the end portion 4 is immediately prepared in a separate working step using a suitable injection molding tool with the hollow needle put in so that the end portion 4 has a still unshown interior thread, so that the end portion 4 can be screwed onto the threaded connector 10 of the cylindrical supply chamber 3. Furthermore the cylindrical supply chamber 3 has a finger support designated with the drawing reference number 9. The cylindrical supply chamber 3 is provided with a piston 21 and a piston rod 23 connected with it so that the piston is axially slidable in the syringe body 2.

In order to make the two part syringe and/or syringe blank shown in FIGS. 2a and/or 2b, the cylindrical supply chamber 3 is made in a first injection molding machine, including the connecting element or connector 10 and the finger support 9. The cylindrical supply chamber is provided at its head end with a connecting element or connector 10, which is preferably a Luerlock adapter.

The end portion 4 is made by a second injection-molding machine separate from the first injection molding machine. After insertion of the hollow needle 6 in the single cavity of this second injection molding machine a thermoplastic material for the end portion 4 is injected around this inserted hollow needle 6. At the same time the inside of the end portion 4 is provided with an interior thread (not shown) so that after removing the end portion 4 provided with the hollow needle 6 it can be screwed immediately on the connector 10.

The disclosure in German Patent Application 102 23 421.3-44 of May 25, 2002 is incorporated here by reference, which describes the invention described hereinabove and claimed in the claims appended hereinbelow and provides the basis for a claim of priority for the instant invention under 35 U.S.C. 119.

While the invention has been illustrated and described as embodied in an injector, especially a pre-filled one-time-use injector, it is not intended to be limited to the details shown, since various modifications and changes may be made without departing in any way from the spirit of the present invention.

Without further analysis, the foregoing will so fully reveal the gist of the present invention that others can, by applying current knowledge, readily adapt it for various applications without omitting features that, from the standpoint of prior art, fairly constitute essential characteristics of the generic or specific aspects of this invention.

What is claimed is new and is set forth in the following appended claims.

We claim:

1. A syringe comprising
a syringe body (2), said syringe body including a cylindrical storage chamber (3) for a liquid to be injected, said cylindrical storage chamber (3) being formed to receive a piston (21) that is axially slidable in the cylindrical storage chamber for dispensing the liquid to be injected;
a finger support or finger gripping element (9) arranged at one end of the syringe body (2);
an end portion (4) arranged at another end of the syringe body (2) opposite from the one end; and
a hollow needle (6) clamped in a needle channel (5) provided in said end portion (4).

2. The syringe as defined in claim 1, wherein said hollow needle (6) is held clamped in said needle channel (5) so that said hollow needle is only removable from said needle channel (5) by applying a force of greater than 30 N to the hollow needle.

3. The syringe as defined in claim 2, wherein said force is between 60 N and 120 N.

4. The syringe as defined in claim 1, wherein an end region of said hollow needle (6) is clamped in a part of the needle channel (5) that is closest to said cylindrical storage chamber (3).

5. The syringe as defined in claim 1, wherein said needle channel (5) clamps the hollow needle (6) over an entire axial length of said needle channel (5).

6. The syringe as defined in claim 1, wherein said end portion (4) is made from thermoplastic material and the hollow needle (6) is clamped in the needle channel (5) by heating and deforming said thermoplastic material.

7. The syringe as defined in claim 1, wherein said end portion (4) provided with the hollow needle (6) is connected with the cylindrical supply chamber (3) by a connector (10).

8. The syringe as defined in claim 7, wherein said connector (10) is threaded to provide a screw connection between the cylindrical supply chamber (3) and the end portion (4).

9. The syringe as defined in claim 1, wherein said end portion (4) is provided with a retaining part for releasably holding a needle protecting device and said retaining part is arranged around an outer periphery of the end portion (4).

10. The syringe as defined in claim 1, wherein said hollow needle is clamped in said needle channel without an adhesive for gluing the hollow needle in the needle channel.

11. The syringe as defined in claim 1, consisting of a pre-filled one-time-use syringe.

12. A syringe comprising
a syringe body (2), said syringe body including a cylindrical storage chamber (3) for a liquid to be injected, a finger support or finger gripping element (9) arranged at one end of the syringe body (2) and an end portion (4) arranged at another end of the syringe body (2) opposite from the one end;
a piston (21) with a piston rod (23) connected with said piston so that said piston (21) is axially slidable in the cylindrical storage chamber (3) for dispensing the liquid to be injected; and
a hollow needle (6) clamped or held fixed in a needle channel (5) provided in the end portion (4) without any adhesive for bonding the hollow needle (6) with the end portion (4).

13. A method of making a syringe body for a syringe, said syringe comprising said syringe body, said syringe body including a cylindrical storage chamber (3) for a liquid to be injected, said cylindrical storage chamber (3) being formed to receive a piston (21) that is axially slidable in the cylindrical storage chamber for dispensing the liquid to be injected, a finger support or finger gripping element (9) arranged at one end of the syringe body (2) and an end portion (4) for holding a hollow needle (6), said end portion (4) being arranged at another end of the syringe body (2) opposite from the one end; wherein the end portion (4) is provided with a needle channel (5) in which the hollow needle (6) is clamped; wherein said method comprises the steps of:
a) first making the cylindrical storage chamber (3); and
b) making the end portion (4) provided with the hollow needle (6) clamped therein; and
c) then after making the end portion (4) and the cylindrical storage chamber (3), connecting the cylindrical storage chamber (3) with the end portion (4) to form the syringe body (2).

14. The method as defined in claim 13, wherein the cylindrical storage chamber (3) is made with a threaded connector formed in one piece therewith by an injection molding process.

15. The method as defined in claim 13, wherein the end portion (4) with the hollow needle (6) clamped therein is made by an injection molding process.

16. A syringe body for a syringe, said syringe body being made by a method comprising the steps of:
a) first making a cylindrical storage chamber (3) in one piece with a threaded connector by an injection molding process; and
b) making an end portion (4) with the hollow needle (6) clamped therein in another injection molding process; and
c) then, after making the end portion (4) and the cylindrical storage chamber (3), connecting the cylindrical storage chamber (3) with the end portion (4).
